(19) United States
(12) Patent Application Publication
(10) Pub. No.: US 2011/0264204 A1
(43) Pub. Date: Oct. 27, 2011

(54) DEVICES AND METHODS FOR DELIVERING AN ENDOCARDIAL DEVICE

(76) Inventor: Alexander Khairkhahan, Palo Alto, CA (US)

(21) Appl. No.: 13/129,961

(22) PCT Filed: Dec. 15, 2009
(86) PCT No.: PCT/US09/67978

§ 371 Date: Jul. 14, 2011

Related U.S. Application Data
(60) Provisional application No. 61/122,678, filed on Dec. 15, 2008.

(51) Int. Cl. A61F 2/24

(52) U.S. Cl. 623/2.11

(57) ABSTRACT

Apical reconstruction and support devices for use in a patient's ventricle include removable implants that may partition the ventricle. Such devices may be implanted using an applicator or system configured for inserting, repositioning and/or removing them. Described herein are applicators, systems, and methods of positioning, deploying and removing cardiac implants. The implants described herein may be inserted into a chamber of a patient's heart, particularly near the apex of the left ventricle, and may support the heart wall. Li some variations the implant is a ventricular partitioning device for partitioning the ventricle into productive and non-productive regions. The applicators may include an expandable member or members at the distal end of a guide to adjustably move the tip of the guide catheter within the ventricle before or during deployment of the implant from the distal end of the guide catheter. These applicators may displace trabeculations within the ventricle.

### CROSS REFERENCE TO RELATED APPLICATIONS

This provisional application claims priority to U.S. provisional patent application Ser. No. 61/122,678, filed on Dec. 15, 2008.

### FIELD OF THE INVENTION

The present invention relates generally to medical/surgical devices and methods pertaining to treating heart disease, particularly congestive heart failure. More specifically, the present invention relates to devices and methods for delivering a partitioning device to a patient's ventricle.

### BACKGROUND OF THE INVENTION

Described herein are systems, methods and devices for improving cardiac function, and may relate generally to treating heart disease, particularly congestive heart failure, and more specifically, to systems, methods, and devices for delivering a partitioning device to a patient's ventricle.

Congestive heart failure annually leads to millions of hospital visits internationally. Congestive heart failure is the description given to a myriad of symptoms that can be the result of the heart's inability to meet the body's demand for blood flow. In certain pathological conditions, the ventricles of the heart become ineffective in pumping the blood, causing a back-up of pressure in the vascular system behind the ventricle.

The reduced effectiveness of the heart is usually due an enlargement of the heart. A myocardial ischemia may, for example, cause a portion of a myocardium of the heart to lose its ability to contract. Prolonged ischaemia can lead to infarction of a portion of the myocardium (heart muscle) wherein the heart muscle dies and becomes scar tissue. Once this tissue dies, it no longer functions as a muscle and cannot contribute to the pumping action of the heart. When the heart tissue is no longer pumping effectively, that portion of the myocardium is said to be hypokinetic, meaning that it is less contractile than the uncompromised myocardial tissue. As this situation worsens, the local area of compromised myocardium may in fact bulge out as the heart contracts, further decreasing the heart's ability to move blood forward. When local wall motion moves in this way, it is said to be dyskinetic, or akinetic. The dyskinetic portion of the myocardium may stretch and eventually form an aneurysmic bulge. Certain diseases may cause a global dilated myopathy, i.e., a general enlargement of the heart when this situation continues for an extended period of time.

As the heart begins to fail, distilling pressures increase, which stretches the ventricular chamber prior to contraction and greatly increases the pressure in the heart. In response, the heart tissue reforms to accommodate the chronically increased filling pressures, further increasing the work that the now comprised myocardium must perform.

Patients suffering from congestive heart failure are commonly grouped into four classes, Classes I, II, III and IV. In the early stages, Classes I and II, drug therapy is presently the most common treatment. Drug therapy typically treats the symptoms of the disease and may slow the progression of the disease, but it cannot cure the disease. Presently, the only permanent treatment for congestive heart disease is heart transplantation, but heart transplant procedures are very risky, extremely invasive and expensive and are performed on a small percentage of patients. Many patient's do not qualify for heart transplant for failure to meet any one of a number of qualifying criteria, and, furthermore, there are not enough hearts available for transplant to meet the needs of CHF patients who do qualify.

Substantial effort has been made to find alternative treatments for congestive heart disease. For example, surgical procedures have been developed to dissect and remove weakened portions of the ventricular wall in order to reduce heart volume. This procedure is highly invasive, risky and expensive and is commonly only done in conjunction with other procedures (such as heart valve replacement or coronary artery by-pass graft). Additionally, the surgical treatment is usually only offered to Class III and IV patients and, accordingly, is not an option for most patients facing ineffective drug treatment. Finally, if the procedure fails, emergency heart transplant is the only presently available option.

Ventricular partitioning devices offer a solution for treating congestive heart disease. These devices generally function to partition a patient's ventricle into a productive region and a non-productive region. For such devices to function properly, they are positioned in a specific location within the patient's heart chamber. Delivery of partitioning device may be made complicated by the presence of anatomical structures within the ventricle of the heart that may prevent the accurate positioning and deployment of the device. For example, the ventricles are known to have trabeculations extending at least partially across the chamber. Thus, it would be beneficial to provide devices, systems and methods for delivering and deploying a partitioning device in a patient's ventricle.

Described herein are treatment delivery devices that are configured to be steerable, and methods of delivering or deploying partitioning devices in a patient's ventricle.

### SUMMARY OF THE INVENTION

Described herein are devices and systems including removable implants, applicators for inserting, repositioning and/or removing them, and methods of positioning, deploying and removing them. The implants described herein are cardiac implants that may be inserted into a chamber of a patient's heart, particularly the left ventricle. The implant may support the heart wall. In some variations the implant is an apical reconstruction or apical protection device. Such implants may also be referred to as a ventricular partitioning device that may partition the ventricle into productive and non-productive regions.

For example, described herein are methods of delivering a ventricular partitioning device to a patient's ventricle, comprising: advancing the distal end of a guide catheter into the patient's ventricle; steering the distal end of the guide catheter within the ventricle by expanding an expandable member at the distal end of the guide catheter to move the tip of the guide catheter within the ventricle; and deploying a ventricular partitioning device from the distal end of the guide catheter so that the ventricular partitioning device expands within the ventricle to partition the ventricle.

Also described herein are methods of delivering a ventricular partitioning device to a patient's heart chamber, comprising: advancing the distal end of a guide catheter into the patient's ventricle; displacing trabeculations within the ventricle by expanding an expandable member at the distal end of the guide catheter; and deploying a ventricular partitioning device from the distal end of the guide catheter so that the ventricular partitioning device expands within the ventricle to partition the ventricle.

Also described herein are systems for delivering a ventricular partitioning device to a patient's ventricle, the system comprising: an elongate guide catheter having: an expandable member at the distal end of the guide catheter configured to steer the distal tip of the guide catheter by expanding; a coupling element at the distal tip of the guide catheter configured to couple a ventricular partitioning device to the guide catheter; and a ventricular partitioning device configured to expand within the patient's ventricle, wherein the ventricular partitioning device includes a plurality of ribs.

Also described herein are systems for delivering a ventricular partitioning device to a patient's ventricle, the system comprising: an elongate guide catheter having: an expandable member at the distal end of the guide catheter configured to steer the distal tip of the guide catheter by expanding; a lumen through the guide catheter having a distal opening, wherein the lumen is configured to pass a partitioning device; and a partitioning device configured to expand within the patient's ventricle, wherein the partitioning device includes a plurality of ribs.

INCORPORATION BY REFERENCE

All publications and patent applications mentioned in this specification are herein incorporated by reference in their entirety to the same extent as if each individual publication or patent application was specifically and individually indicated to be incorporated by reference.

### BRIEF DESCRIPTION OF THE DRAWINGS

FIGS. 1A and 1B are drawings of an embodiment of the system for delivering a ventricular partitioning device.

FIGS. 2A and 2B are drawings of an embodiment of the system for delivering a ventricular partitioning device.

FIGS. 3A and 3B are drawings of an embodiment of the system for delivering a ventricular partitioning device with a plurality of expandable members.

FIGS. 3C-3F are drawings of an expanded expandable member at the distal end of the guide catheter to move the tip of the guide catheter within the ventricle.

FIG. 4 is a drawing of an embodiment of the system for delivering a ventricular partitioning device.

FIGS. 5A-5E are drawings demonstrating a method of delivering a ventricular partitioning device to a patient's ventricle.

FIG. 6 is a drawing of an embodiment of the ventricular partitioning device.

### DETAILED DESCRIPTION OF THE INVENTION

Various embodiments of devices and systems, as well as methods, for delivering and/or deploying a partitioning device into a patient's ventricle are described herein. The following description is not intended to limit the invention to these embodiments, but rather to enable any person skilled in the art to make and use this invention.

In general, the devices and systems described herein include an expandable member at the distal end of the guide catheter configured to steer the distal tip of the guide catheter by expanding and a coupling element at the distal tip of the guide catheter configured to couple a ventricular partitioning device. The guide catheter may also include a handle and one or more controls. In addition, the expandable member may be an inflatable member (e.g., a balloon) or a plurality of expandable members. For example, a plurality of expandable members may be arranged around a distal perimeter of the guide catheter to help position a ventricular partitioning device attached thereto.

### Systems for Delivering a Ventricular Partitioning Device

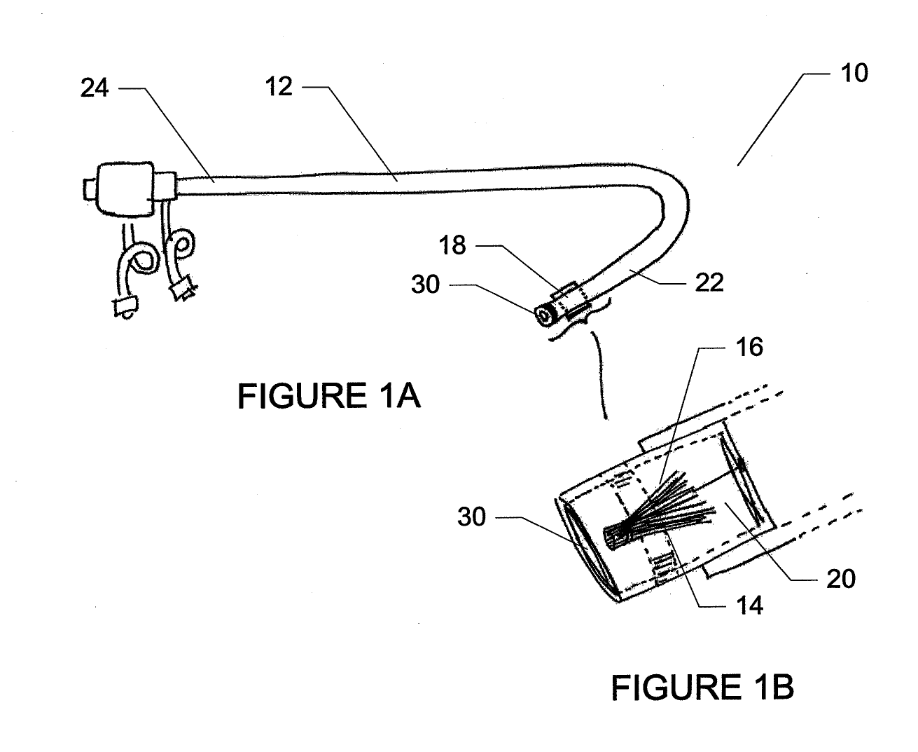

FIGS. 1A and 1B illustrate one system 10 for delivering a ventricular partitioning device to a patient's ventricle. This system includes an elongate guide catheter 12 and a ventricular partitioning device 14, having a plurality of ribs 16, configured to expand within the patient's ventricle. The elongate guide catheter includes an expandable member 18, at the distal end of the guide catheter that functions to steer the distal tip of the guide catheter by expanding, and a coupling element 20 at the distal tip of the guide catheter that functions to couple a ventricular partitioning device to the guide catheter. The system is designed to deliver a ventricular partitioning device to a patient's ventricle. The device may be alternatively used to deliver a device to any other suitable chamber or organ of a patient, or deliver a device in any other suitable environment, and for any suitable purpose.

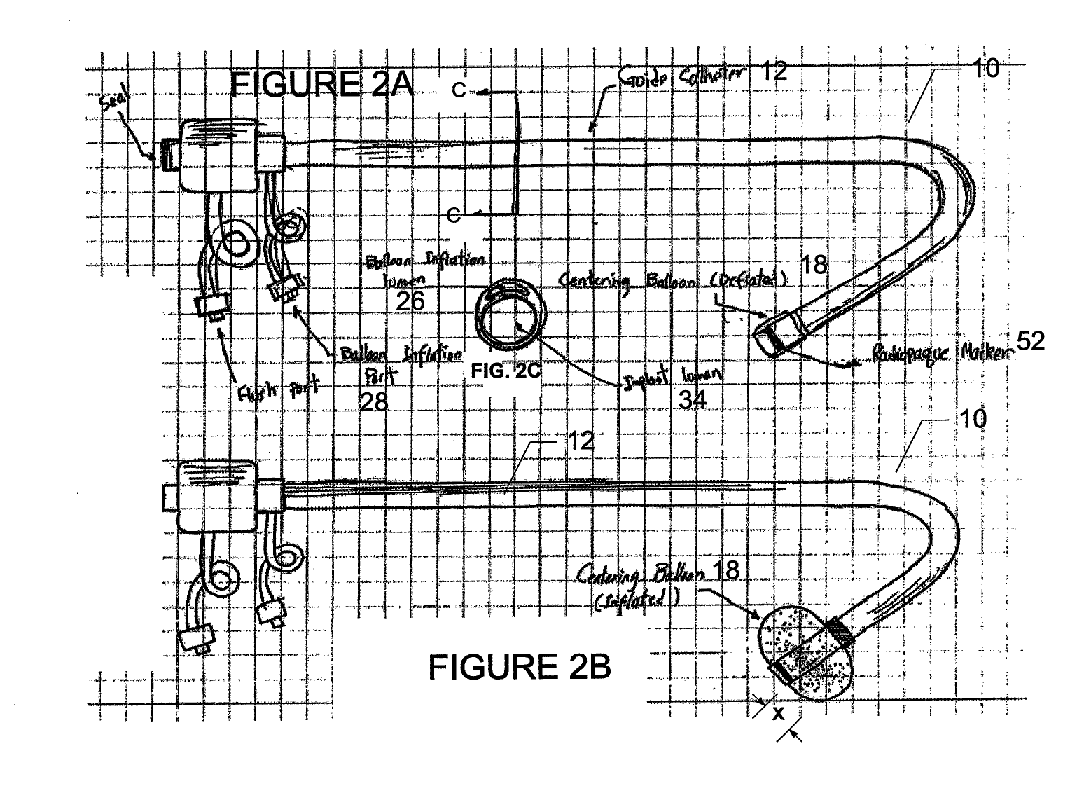

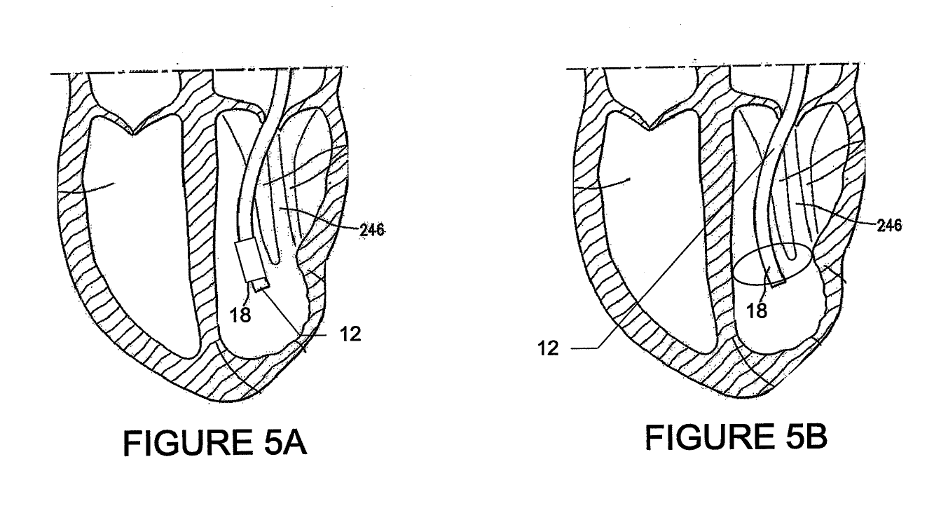

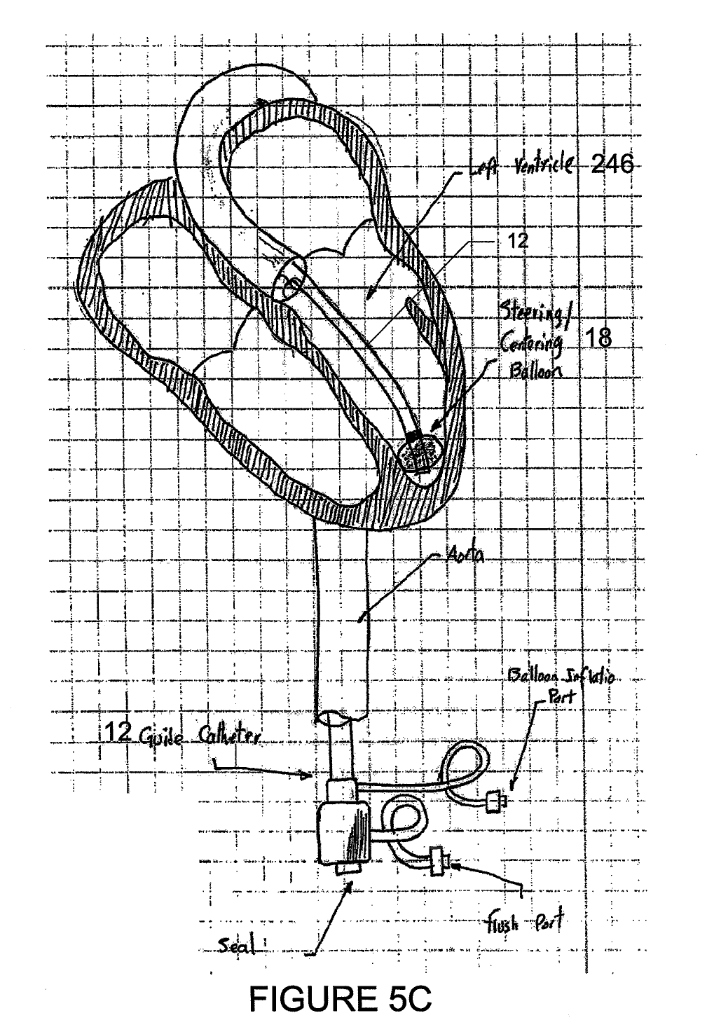

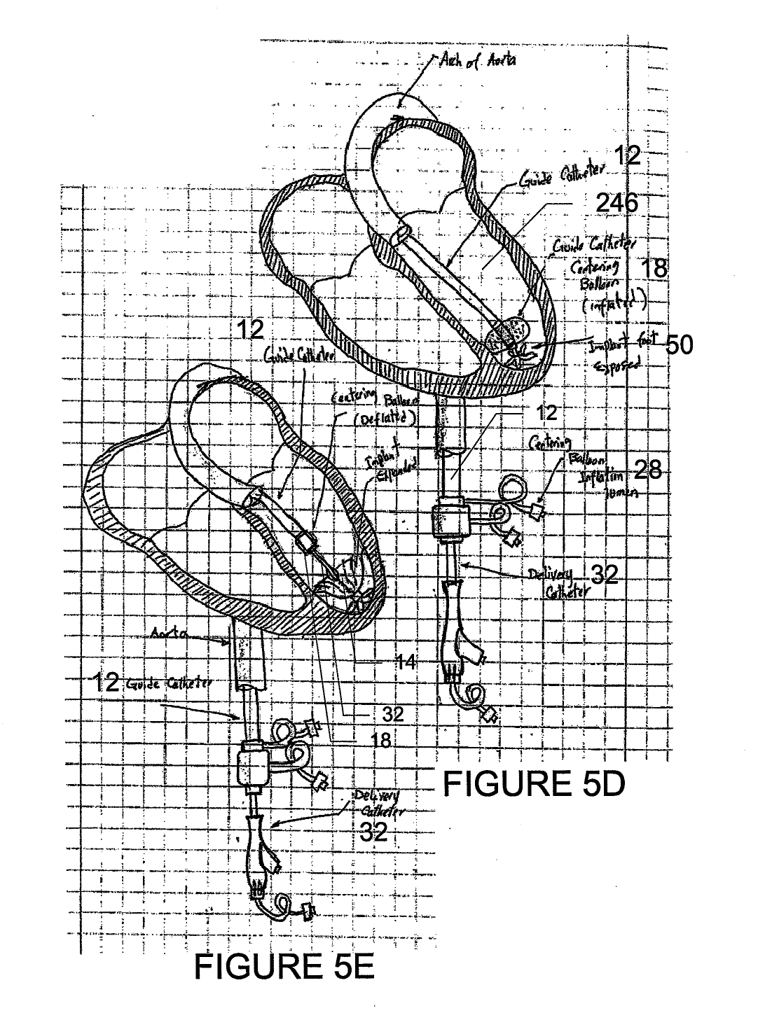

As shown in FIGS. 1A and 1B, the elongate guide catheter 12 functions to transport the ventricular partitioning device 14. The elongate guide catheter has a distal portion 22 and a proximal portion 24. The distal portion may be advanced into the patient, while the proximal portion may remain exterior to the patient. In some embodiments, the distal portion of the elongate guide catheter is configured to be inserted through the aorta and aortic valve, and into the left ventricle (as shown in FIG. 5C). Additionally, the distal portion of the elongate guide catheter may be configured to be percutaneously introduced into the patient's vasculature, and then advanced through the vasculature to the aorta, or any other suitable vessel. In some embodiments, the distal portion of the elongate guide catheter is configured to transport the ventricular partitioning device into the ventricle through the distal wall of the ventricle. As shown in FIG. 2A, in some embodiments, the guide catheter further includes a marker 52 at the distal end of the guide catheter 12. The marker may allow the guide catheter to be visualized while it is advanced and/or placed within the patient. The marker may be configured to be visible by eye, ultrasound, X-ray, or any other suitable visualization techniques. In some embodiments, the markers are radio-opaque and made from stainless steel, platinum, gold, iridium, tantalum, tungsten, silver, rhodium, nickel, bismuth, or any other suitable metal, alloy, or oxide of these metals.

The elongate guide catheter may have a circular cross section, or may alternatively have any other suitable cross section. The guide catheter may be hollow or tubular along the entire the length of the catheter, or alternatively, a portion of the length of the catheter may be hollow. The guide catheter may have a single lumen or multiple lumens. In some embodiments, the guide catheter is made from a flexible material and has a length greater than 100 cm and a diameter smaller than 10 mm. In one specific embodiment, the guide catheter has a length of 110 cm and a diameter of 5 mm.

As shown in FIGS. 2A and 2B, an elongate guide catheter 12 may include an expandable member 18 at the distal end of the guide catheter. The expandable member may steer the distal tip of the guide catheter by expanding (as shown in FIG. 2B). More specifically, the expandable member may be configured to expand against a portion of the ventricle, thereby moving the tip of the guide catheter away from the portion of the ventricle. For example, a guide catheter may be inserted into a ventricle of a patient such that the guide catheter is in contact with the inner wall of the ventricle. The expandable member may be expanded to a dimension (labeled x in FIG. 2B) beyond the outer diameter of the guide catheter. The expandable member, as it is expanded, contacts the inner wall of the ventricle and moves the distal portion of the guide catheter to a distance away from the inner wall of the ventricle. The distance may be equal to or greater than the dimension of the expandable member.

In some embodiments, as shown in FIG. 2A and 2B, the expandable member 18 is a balloon. To expand, the balloon may be filled with an inflation fluid such as saline, air, water, or any other suitable fluid. The expandable member may be deflated by releasing or removing the fluid from the balloon. In this embodiment, the elongate guide catheter 12 preferably includes an inflation lumen 26 and an inflation port 28. The inflation port may be located proximal to the expandable member (i.e. at the proximal end of the elongate guide catheter), and functions to receive the inflation fluid. In this example, the inflation lumen is disposed within the guide catheter, along the length of the catheter from the inflation port to the expandable member. The interior of the expandable member is in fluid communication with the inflation lumen, which is in fluid communication with the inflation port. As shown in FIG. 2C, the inflation lumen 26 may have any suitable cross section such as crescent shaped or circular. Additionally, the inflation lumen may have a different cross sectional diameter than other lumen(s) within the guide catheter. For example, as shown in FIG. 2C, the guide catheter may include a lumen 34 through which a delivery catheter is disposed and/or through which the ventricular partitioning device is delivered.

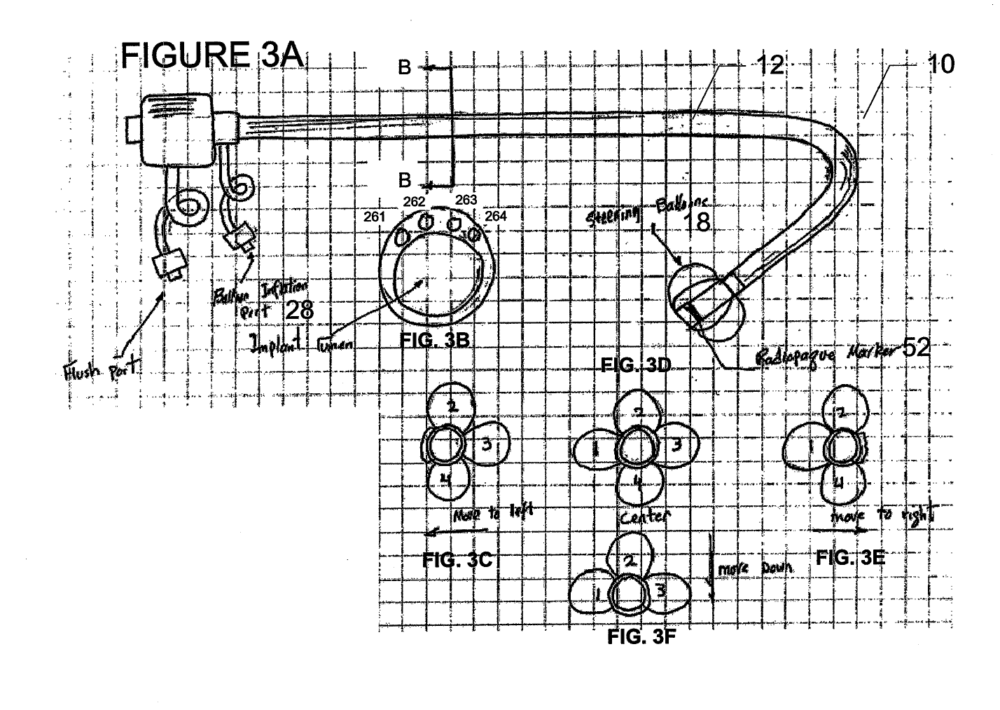

In some embodiments, as shown in FIG. 3A, the elongate guide catheter 12 includes a plurality of expandable members. As shown in FIGS. 3C-3F, the guide catheter includes four expandable members 1, 2, 3, and 4 arranged around the circumference of the elongate guide catheter; alternatively the guide catheter may include any other suitable number of expandable members, arranged in any suitable configuration. The plurality of expandable members function to steer the distal tip of the guide catheter by expanding (FIGS. 3C-3F). More specifically, each expandable member may be configured to expand against a portion of the ventricle, thereby moving the tip of the guide catheter away from that portion of the ventricle. For example, as shown in FIG. 3C, to move the catheter tip to the “left”, or in the direction away from expandable member 3, expandable member 3 is expanded such that the guide catheter moves away from the inner wall of the ventricle in the direction shown by the arrow. As shown in FIG. 3D, in order to center the device, each of the expandable members 1, 2, 3, and 4 may be expanded. As shown in FIG. 3F, to move the catheter tip “down”, or in the direction away from expandable member 2, expandable member 2 is expanded such that the guide catheter moves away from the inner wall of the ventricle in the direction shown by the arrow.

As shown in FIG. 3A and 3B, in some embodiments, the plurality of expandable members is a plurality of balloons. In this embodiment, the elongate guide catheter 12 preferably includes a plurality of inflation lumens 261, 262, 263, 264 and an inflation port 28 or ports. The interior of each of the expandable members is in fluid communication with each of the inflation lumens. For example, inflation lumen 261 may function to deliver inflation fluid from the inflation port to expandable member 1. As shown in FIG. 3B, the inflation lumens may have any suitable cross section such as circular. Each of the plurality of inflation lumens may have the same size and shape, or may alternatively have differing sizes and shapes.

In some embodiments, the plurality of expandable members is a plurality of flexible ribs. The plurality of flexible ribs may be arranged around the circumference of the guide catheter, or may alternatively be arranged in any other suitable configuration. In some embodiments, the expandable member may include a membrane, coupled to the flexible ribs and/or coupling the flexible ribs to one another. In the non-expanded configuration, the ribs may be substantially flush to the outer surface of the guide catheter. The ribs expand by bending or bowing away from the outer diameter of the guide catheter. In a first variation, the flexible ribs are a shape memory material. In a second variation, the flexible ribs are configured such that a first movable end of the rib is moved towards a second fixed end of the rib such that the ribs bend and bow away from the outer diameter of the guide catheter.

As described above, the expandable member may be configured to displace trabeculations (and in some variations chordae tendinae/papillary muscles) within the ventricle. The ventricles of most patients have numerous trabeculations, or muscular projections, connecting various portions of the inner wall of the ventricle to one another. The expandable member, in its expanded state, functions to atraumatically push through or displace the trabeculations. Furthermore, once expanded, the expandable member increases the overall diameter of the distal portion of the guide catheter, thereby preventing the guide catheter from becoming trapped or tangled among the trabeculations of the ventricle.

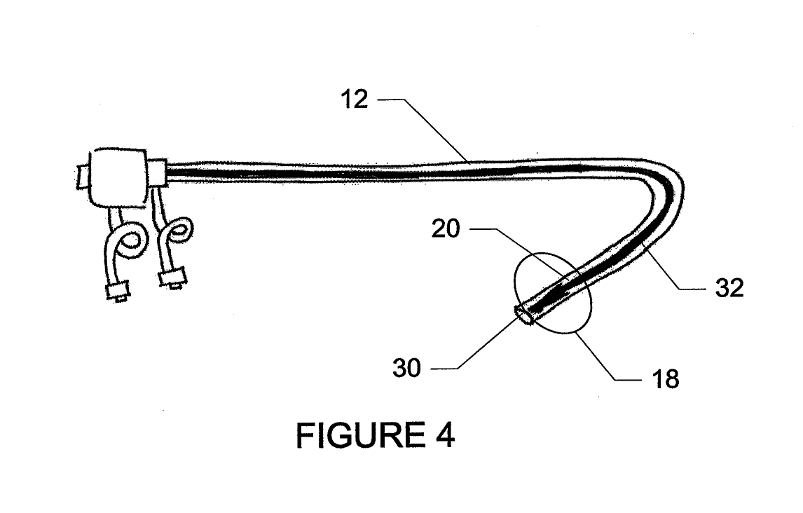

As shown in FIGS. 1A, 1B, and 4, the elongate guide catheter 12 may include a coupling element 20 at the distal tip of the guide catheter that functions to couple a ventricular partitioning device to the guide catheter. In some embodiments, the coupling element may be an opening 30 at the distal tip configured to pass a partitioning device. As shown in FIG. 1B, the opening may be a lumen or hollow portion of the guide catheter that extends along a portion of the guide catheter. Alternatively, as shown in FIG. 4, the lumen may run the length of the guide catheter. In some embodiments, as shown in FIG. 4, the guide catheter further includes a delivery catheter 32 that functions to pass through a lumen of the guide catheter and release the ventricular partitioning device 14 from the opening 30 at the distal end of the guide catheter. As shown in FIG. 5E, the delivery catheter 32 is disposed within the guide catheter, along the length of the guide catheter 12, and is coupled to the partitioning device 14. The delivery catheter functions to push the partitioning device through the guide catheter (and/or opening 30), or otherwise release the partitioning device. In some embodiments, the delivery catheter is slidable within the lumen of the guide catheter. For example, as shown in FIG. 2C, the delivery catheter may be disposed within the guide catheter within the lumen 34.

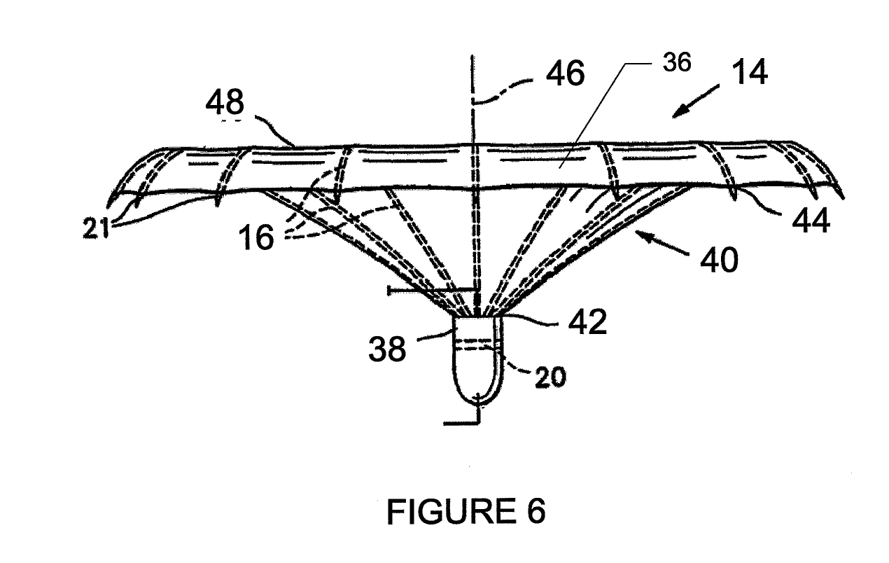

As shown in FIG. 6, the ventricular partitioning device 14 functions to partition a ventricle of a patient's heart into a productive region and a non-productive region. The partitioning device may be one of several variations. In a first variation, the ventricular partitioning device includes a partitioning membrane 36, a hub 38, preferably centrally located on the partitioning device, and a radially expandable reinforcing frame 40 formed of a plurality of ribs 16. In some embodiments, the hub may include feet 50 (as shown in FIG. 5D) or any other suitable pedestal to atraumatically couple the partitioning device to the chamber wall. Alternatively, the hub may include an anchor (not shown) that functions to couple the device to the tissue of the heart chamber and secure the partitioning device. The partitioning membrane 36 may be secured to the proximal or pressure side of the frame 40 as shown in FIG. 6. The ribs have distal ends 42 which are secured to the hub and free proximal ends 44 which may be configured to curve or flare away from a center line axis 46. Radial expansion of the free proximal ends unfurls the membrane secured to the frame so that the membrane presents a relatively smooth, pressure receiving surface 48 which defines in part the productive portion of the patient's partitioned heart chamber. In some embodiments, the curved free proximal ends of ribs may be provided with sharp tip elements 21 which are configured to hold the frame and the membrane secured thereto in a deployed position within the patient's heart chamber. The sharp tip elements of the frame penetrate into tissue of the patient's heart wall in order to secure the partitioning device within the heart chamber so as to partition the ventricular chamber into a productive region (proximal to the device) and a non-productive region (distal to the device). The ventricular partitioning device may alternatively, have any other suitable configuration, including any suitable combination of elements (ribs, membranes, sharp tip elements, hubs, feet, anchors, etc.), such that it functions to partition the ventricular chamber into a productive region (proximal to the device) and a non-productive region (distal to the device).

### Methods of Delivering a Ventricular Partitioning Device

As shown in FIGS. 5A-5E, the method of delivering a ventricular partitioning device to a patient's ventricle includes the steps of advancing the distal end of a guide catheter 12 into the patient's ventricle 246 (FIG. 5A), steering the distal end of the guide catheter within the ventricle by expanding an expandable member 18 at the distal end of the guide catheter to move the tip of the guide catheter within the ventricle (FIGS. 5B and 5C), and deploying a ventricular partitioning device 14 from the distal end of the guide catheter so that the ventricular partitioning device expands within the ventricle to partition the ventricle (FIGS. 5D and 5E). The method is designed to deliver a ventricular partitioning device to a patient's ventricle. The method may be alternatively used to deliver a device to any other suitable chamber or organ of a patient, or deliver a device in any other suitable environment, and for any suitable purpose.

As shown in FIG. 5A, the step of advancing the distal end of a guide catheter into the patient's ventricle functions to position the distal end of the guide catheter into the ventricle such that the guide catheter transports the ventricular partitioning device to the ventricle. In some embodiments, the distal portion of the elongate guide catheter may be advanced through the aorta and aortic valve, and into the left ventricle. Additionally, the distal portion of the elongate guide catheter may first be advanced percutaneously into the patient's vasculature, and then advanced through the vasculature to the aorta, or any other suitable vessel. In some embodiments, the distal portion of the elongate guide catheter may be advanced into the ventricle through the distal wall of the ventricle. In some embodiments, the step of advancing the distal end of a guide catheter into the patient's ventricle includes advancing the ventricular partitioning device, coupled to the guide catheter, in a contracted configuration (FIG. 1B).

As shown in FIGS. 5B and 5C, the step of steering the distal end of the guide catheter within the ventricle by expanding an expandable member at the distal end of the guide catheter to move the tip of the guide catheter within the ventricle functions to position the tip of the guide catheter into a desired location for the deployment of the ventricular partitioning device (deployment of device described in detail below). The step of steering the distal end of the guide catheter within the ventricle by expanding an expandable member includes expanding the expandable member against a portion of the ventricle, thereby moving the tip of the guide catheter away from the portion of the ventricle. For example, a guide catheter may be inserted into a ventricle of a patient such that the guide catheter is in contact with the inner wall of the ventricle. The expandable member may be expanded to a dimension (labeled x in FIG. 2B) beyond the outer diameter of the guide catheter. The expandable member, as it is expanded, may contact the inner wall of the ventricle, and move the distal portion of the guide catheter to a distance away from the inner wall of the ventricle. The distance may be the same size or greater than the dimension of the expandable member. The step of steering the distal end of the guide catheter within the ventricle includes steering the distal end of the catheter towards a target region within the ventricle, such as towards the apex of the ventricle, i.e. the distal tip of the ventricle. The catheter may be steered towards the center of the target region, or offset from the center of the target region.

In some embodiments, as shown in FIGS. 3C-3F, the step of steering the distal end of the guide catheter within the ventricle by expanding the expandable member includes expanding a plurality of expandable members. As shown in FIG. 3C, the step of steering the distal end of the guide catheter within the ventricle by expanding the expandable member includes expanding expandable member 3 and contacting a portion of the ventricle with the expandable member 3, thereby moving the tip of the guide catheter toward expandable member 1 (shown not expanded) on an opposing side of the guide catheter from expandable member 3. As shown in FIG. 3E, the step of steering the distal end of the guide catheter within the ventricle by expanding the expandable member further includes expanding expandable member 1 on the opposing side of the guide catheter from expandable member 3 and contacting a second portion of the ventricle with expandable member 1, thereby moving the tip of the guide catheter toward the expandable member 3. The same method may alternatively be used to with any other suitable combinations of expandable members, for example by expanding all four expandable members, as shown in FIG. 3D, the expandable members function to center the distal tip of the catheter with respect to the walls of the ventricle.

In some embodiments, as may be shown, the step of steering the distal end of the guide catheter within the ventricle further includes displacing trabeculations within the ventricle. The step of displacing trabeculations within the ventricle includes expanding the expandable member against the trabeculations. The expandable member, in its expanded state, functions to atraumatically push through or displace the trabeculations. Furthermore, once expanded, the expandable member increases the overall diameter of the distal portion of the guide catheter, thereby protecting the guide catheter from becoming trapped or tangled among the trabeculations of the ventricle.

As shown in FIGS. 5D and 5E, the step of deploying a ventricular partitioning device from the distal end of the guide catheter so that the ventricular partitioning device expands within the ventricle to partition the ventricle functions to release the partitioning device from the guide catheter in the desired location within the ventricle such that the partitioning device forms a productive region and a non-productive region of the heart. In some embodiments, as shown in FIG. 5D, the step of deploying the ventricular partitioning device includes extending the ventricular partitioning device from within a lumen at the distal end of the guide catheter. In some embodiments, as shown in FIG. 5E, the method further includes the step of advancing a delivery catheter 32 through a lumen of the guide catheter. In this embodiment, the ventricular partitioning device may be released from the delivery catheter.

In some embodiments, the method of delivering a ventricular partitioning device to a patient's ventricle includes the steps of advancing the distal end of a guide catheter 12 into the patient's ventricle (not shown), displacing trabeculations within the ventricle by expanding an expandable member at the distal end of the guide catheter (not illustrated), and deploying a ventricular partitioning device from the distal end of the guide catheter so that the ventricular partitioning device expands within the ventricle to partition the ventricle.

As may be shown, the step of displacing trabeculations within the ventricle by expanding an expandable member at the distal end of the guide catheter functions to facilitate the movement of the guide catheter through a series of trabeculations and beyond the trabeculations to a more distal portion of the ventricle. The step of displacing the trabeculations within the ventricle includes expanding the expandable member against the trabeculations. The expandable member, in its expanded state, functions to atraumatically push through or displace the trabeculations. Furthermore, once expanded, the expandable member increases the overall diameter of the distal portion of the guide catheter, thereby protecting the guide catheter from becoming trapped or tangled among the trabeculations of the ventricle.

Once the guide catheter has displaced and/or moved through the trabeculations, in some embodiments, the method further includes the step of steering the distal end of the guide catheter within the ventricle by expanding an expandable member at the distal end of the guide catheter to move the tip of the guide catheter within the ventricle as shown in FIGS. 5B and 5C. The step of steering the distal end of the guide catheter within the ventricle by expanding an expandable member includes expanding the expandable member against a portion of the ventricle, thereby moving the tip of the guide catheter away from the portion of the ventricle. The step of steering the distal end of the guide catheter within the ventricle includes steering the distal end of the catheter towards a target region within the ventricle, such as towards the apex of the ventricle, i.e. the distal tip of the ventricle. The catheter may be steered towards the center of the target region, or offset from the center of the target region.

While particular forms of the invention have been illustrated and described herein, it will be apparent that various modifications and improvements can be made to the invention. Moreover, individual features of embodiments of the invention may be shown in some drawings and not in others, but those skilled in the art will recognize that individual features of one embodiment of the invention can be combined with any or all the features of another embodiment. Accordingly, it is not intended that the invention be limited to the specific embodiments illustrated. It is intended that this invention to be defined by the scope of the appended claims as broadly as the prior art will permit.

What is claimed is:

1. A method of delivering a ventricular partitioning device to a patient's ventricle, comprising:
advancing the distal end of a guide catheter into the patient's ventricle;
steering the distal end of the guide catheter within the ventricle by expanding an expandable member at the distal end of the guide catheter to move the tip of the guide catheter within the ventricle; and
deploying a ventricular partitioning device from the distal end of the guide catheter so that the ventricular partitioning device expands within the ventricle to partition the ventricle.

2. The method of claim 1, wherein the step of advancing the distal end of a guide catheter into the patient's ventricle comprises advancing the ventricular partitioning device, coupled to the guide catheter, in a contracted configuration.

3. The method of claim 1, wherein the step of steering the distal end of the guide catheter within the ventricle comprises steering the distal end of the guide catheter to a target region within the ventricle.

4. The method of claim 3, wherein the step of steering the distal end of the guide catheter within the ventricle further comprises centering the tip of the guide catheter relative to the target region.

5. The method of claim 3, wherein the step of steering the distal end of the guide catheter to a target region within the ventricle comprises steering the distal end of the guide catheter toward the apex of the ventricle.

6. The method of claim 1, wherein the step of steering the distal end of the guide catheter within the ventricle comprises displacing trabeculations within the ventricle.

7. The method of claim 6, wherein the step of steering the distal end of the guide catheter within the ventricle comprises expanding the expandable member against the trabeculations.

8. The method of claim 1, wherein expanding an expandable member comprises inflating a balloon.

9. The method of claim 1, wherein the step of steering the distal end of the guide catheter within the ventricle by expanding the expandable member comprises expanding a plurality of expandable members.

10. The method of claim 1, wherein the step of steering the distal end of the guide catheter within the ventricle by expanding the expandable member comprises expanding a plurality of expandable members that are arranged around the perimeter of the tip of the guide catheter.

11. The method of claim 1, wherein the step of steering the distal end of the guide catheter within the ventricle by expanding the expandable member comprises expanding a first balloon and contacting a first portion of the ventricle with the first balloon, thereby moving the tip of the guide catheter toward a second balloon on an opposing side of the guide catheter from the first balloon.

12. The method of claim 11, further comprising expanding the second balloon on the opposing side of the guide catheter from the first balloon and contacting a second portion of the ventricle with the second balloon, thereby moving the tip of the guide catheter toward the first balloon.

13. The method of claim 1, wherein the step of deploying the ventricular partitioning device comprises extending the ventricular partitioning device from within a lumen at the distal end of the guide catheter.

14. The method of claim 1, wherein the step of deploying the ventricular partitioning device comprises forming a productive region and a non-productive region of the ventricle with the ventricular partitioning device.

15. The method of claim 1, further comprising the step of advancing a delivery catheter through a lumen of the guide catheter, wherein the ventricular partitioning device is configured to be released from the delivery catheter.

16. A method of delivering a ventricular partitioning device to a patient's heart chamber, comprising:
advancing the distal end of a guide catheter into the patient's ventricle;
displacing trabeculations within the ventricle by expanding an expandable member at the distal end of the guide catheter; and
deploying a ventricular partitioning device from the distal end of the guide catheter so that the ventricular partitioning device expands within the ventricle to partition the ventricle.

17. The method of claim 16, wherein the step of advancing the distal end of a guide catheter into the patient's ventricle comprises advancing the ventricular partitioning device, coupled to the guide catheter, in a contracted configuration.

18. The method of claim 16, wherein the step of displacing trabeculations within the ventricle comprises atraumatically displacing trabeculations within the ventricle and advancing the distal end of the guide catheter further into the patient's ventricle.

19. The method of claim 16, wherein the step of displacing trabeculations within the ventricle by expanding an expandable member comprises inflating a balloon.

20. The method of claim 16, wherein the step of displacing trabeculations within the ventricle by expanding the expandable member comprises expanding a plurality of expandable members.

21. The method of claim 20, wherein the step of displacing trabeculations within the ventricle by expanding an expandable member comprises expanding a plurality of expandable members that are arranged around the perimeter of the tip of the guide catheter.

22. The method of claim 16, wherein the step of displacing trabeculations within the ventricle further comprises steering the distal end of the guide catheter within the ventricle to move the tip of the guide catheter within the ventricle.

23. The method of claim 16, wherein the step of displacing trabeculations within the ventricle further comprises steering the distal end of the guide catheter within the ventricle to a target region within the ventricle.

24. The method of claim 23, wherein the step of steering the distal end of the guide catheter within the ventricle further comprises centering the tip of the guide catheter relative to the target region.

25. The method of claim 16, wherein the step of deploying the ventricular partitioning device comprises extending the ventricular partitioning device from within a lumen at the distal end of the guide catheter.

26. The method of claim 16, wherein the step of deploying the ventricular partitioning device comprises forming a productive region and a non-productive region of the ventricle with the ventricular partitioning device.

27. The method of claim 16, further comprising the step of advancing a delivery catheter through a lumen of the guide catheter, wherein the ventricular partitioning device is configured to be released from the delivery catheter.

28. A system for delivering a ventricular partitioning device to a patient's ventricle, the system comprising:
an elongate guide catheter having:
an expandable member at the distal end of the guide catheter configured to steer the distal tip of the guide catheter by expanding;
a coupling element at the distal tip of the guide catheter configured to couple a ventricular partitioning device to the guide catheter; and
a ventricular partitioning device configured to expand within the patient's ventricle, wherein the ventricular partitioning device includes a plurality of ribs.

29. The system of claim 28, wherein the expandable member is a balloon.

30. The system of claim 28, wherein the guide catheter further includes an inflation lumen and an inflation port; wherein the inflation port is proximal to the distal end of the guide catheter and in fluid communication with the inflation lumen for delivery of inflation fluid therethrough to the interior of the expandable member.

31. The system of claim 28, wherein the guide catheter includes a plurality of expandable members.

32. The system of claim 31, wherein the plurality of expandable members are arranged around the circumference of the distal tip of the catheter.

33. The system of claim 31, wherein the guide catheter further includes a plurality of inflation lumens and a plurality of inflation ports proximal to the distal end of the elongate guide catheter; wherein each inflation port is in fluid communication with an inflation lumen for delivery of inflation fluid therethrough to the interior of each of the balloons.

34. The system of claim 28, wherein the expandable member is configured to displace trabeculations within the ventricle.

35. The system of claim 28, wherein the coupling element is an opening at the distal tip configured to pass a partitioning device.

36. The system of claim 28, further comprising a delivery catheter configured to pass through a lumen in the guide catheter and release the ventricular partitioning element from an opening forming the coupling element at the distal end of the guide catheter.

37. The system of claim 28, wherein the guide catheter further includes a radio opaque marker at the distal end of the guide catheter.

38. A system for delivering a ventricular partitioning device to a patient's ventricle, the system comprising:
an elongate guide catheter having:
an expandable member at the distal end of the guide catheter configured to steer the distal tip of the guide catheter by expanding;
a lumen through the guide catheter having a distal opening, wherein the lumen is configured to pass a partitioning device; and
a partitioning device configured to expand within the patient's ventricle, wherein the partitioning device includes a plurality of ribs.

39. The system of claim 38, wherein the expandable member is a balloon.

40. The system of claim 38, wherein the expandable member is configured to expand against a portion of the ventricle, thereby moving the tip of the guide catheter away from the portion of the ventricle.

41. The system of claim 38, wherein the guide catheter further includes an inflation lumen and an inflation port; wherein the inflation port is proximal to the distal end of the guide catheter and in fluid communication with the inflation lumen for delivery of inflation fluid therethrough to the interior of the expandable member.

42. The system of claim 38, wherein the guide catheter includes a plurality of expandable members.

43. The system of claim 38, further comprising a plurality of expandable members arranged around the circumference of the distal tip of the catheter.

44. The system of claim 38, wherein the guide catheter further includes a plurality of inflation lumens and a plurality of inflation ports proximal to the distal end of the elongate guide catheter; wherein each inflation port is in fluid communication with an inflation lumen for delivery of inflation fluid therethrough to the interior of each of the balloons.

45. The system of claim 38, wherein the expandable member is configured to displace trabeculations within the ventricle.

46. The system of claim 38, further comprising a delivery catheter configured to be slidable within the lumen of the guide catheter, wherein the delivery catheter is configured to release the ventricular partitioning element.

47. The system of claim 38, wherein the guide catheter further includes a radio opaque marker at the distal end of the guide catheter.
